(19) United States
(12) Patent Application Publication
(10) Pub. No.: US 2021/0345886 A1
(43) Pub. Date: Nov. 11, 2021

(54) SKIN TEMPERATURE SCANNING VISUAL SELF ALIGNMENT

(71) Applicant: Litens Health Inc., Woodbridge (CA)

(72) Inventors: Geoffrey William RYELAND, North York (CA); Gary John SPICER, Mississauga (CA); Zhengjie JIA, Woodbridge (CA)

(21) Appl. No.: 17/307,220

(22) Filed: May 4, 2021

Related U.S. Application Data
(60) Provisional application No. 63/021,650, filed on May 7, 2020.
(60) Provisional application No. 63/048,646, filed on Jul. 6, 2020.

(51) Int. Cl. A61B 5/01; A61B 5/16

(52) U.S. Cl. CPC A61B 5/01; A61B 5/163

(57) ABSTRACT

In an aspect, there is provided a temperature scanner for measuring a temperature of a selected area on a human subject having a first eye and a second eye. The temperature scanner includes an infra-red sensor, a first reflective surface region, a second reflective surface region, and a controller. The infra-red sensor has a selected field of view so as to detect a temperature of the selected area of the human subject when the selected area is positioned to completely fill the field of view. The first reflective surface region is concave, has a first principal axis, and has a first focal point along the first principal axis. The first reflective surface region and the second reflective surface region are positioned relative to the infra-red sensor such that positioning the first eye of the human subject at a selected first eye position and positioning the second eye of the human subject at a selected second eye position positions the selected area of the human subject to completely fill the field of view. The controller is configured to receive input from the infra-red sensor that is indicative of a temperature measured by the infra-red sensor. The controller is configured to transmit to an output device an output that is indicative of the temperature measured by the infra-red sensor.

### CROSS-REFERENCE TO RELATED APPLICATIONS

This application claims the benefit of U.S. Provisional Application No. 63/021,650, filed May 7, 2020 and U.S. Provisional Application No. 63/048,646, filed Jul. 6, 2020, the contents of both of which are incorporated herein by reference in their entireties.

### FIELD

The specification relates generally to non-contact temperature measurement devices for people and more particularly for ways to ensure that a person is positioned correctly in order for their temperature to be measured.

### BACKGROUND OF THE DISCLOSURE

It is desirable currently to permit people to take their temperature using a non-contact temperature measurement device, in order to determine if their temperature is elevated, since this can be indicative that they have a fever and may therefore be harboring a virus and may be contagious. It is known currently to mount a non-contact temperature measurement device for use by people wishing to enter a building or a business. However, some such devices are relatively complex and require sophisticated software in order to ensure that the person whose temperature is being taken is positioned correctly. As a result, such devices are typically only purchasable by large, relatively wealthy organizations.

It would be desirable to provide a non-contact temperature measurement device that is relatively inexpensive.

### SUMMARY OF THE DISCLOSURE

In one aspect, there is provided a temperature scanner for measuring a temperature of a selected area on a human subject having a first eye and a second eye. The temperature scanner includes an infra-red sensor, a first reflective surface region, a second reflective surface region, and a controller. The infra-red sensor has a selected field of view so as to detect a temperature of the selected area of the human subject when the selected area is positioned to completely fill the field of view. The first reflective surface region is concave, has a first principal axis, and has a first focal point along the first principal axis. The first reflective surface region and the second reflective surface region are positioned relative to the infra-red sensor such that positioning the first eye of the human subject at a selected first eye position and positioning the second eye of the human subject at a selected second eye position positions the selected area of the human subject to completely fill the field of view. The controller is configured to receive input from the infra-red sensor that is indicative of a temperature measured by the infra-red sensor. The controller is configured to transmit to an output device an output that is indicative of the temperature measured by the infra-red sensor.

In another aspect, there is provided a temperature scanner for measuring a temperature of a selected area on a human subject having a first eye and a second eye. The temperature scanner includes an infra-red sensor, a first reflective surface region, a second reflective surface region, a first set of first positional indicia and a second set of second positional indicia, and a controller. The infra-red sensor has a selected field of view so as to detect a temperature of the selected area of the human subject when the selected area is positioned to completely fill the field of view. The first reflective surface region and the second reflective surface region are positioned relative to the infra-red sensor, such that positioning the first eye of the human subject at a selected first eye position and positioning the second eye of the human subject at a selected second eye position positions the selected area of the human subject to completely fill the field of view. At the selected first eye position the first eye of the human subject can see a first reflected image of the human subject in the first reflective surface region, and at the selected second eye position the second eye of the human subject can see a second reflected image of the human subject in the second reflective surface region. In some embodiments, the first reflected image is a reflected image of the first eye of the human subject, and the second reflected image is a reflected image of the second eye of the human subject. In other embodiments, the first reflected image is a reflected image of some other portion of the human subject other than the first eye, and the second reflected image is a reflected image of some other portion of the human subject other than the second eye. The first positional indicia and second positional indicia are positioned relative to the first and second reflective surface regions. Positioning of the first and second eyes of the human subject so as to align the first and second reflected images with the first and second positional indicia positions the first eye of the human subject in the selected first eye position and positions the second eye of the human subject in the selected second eye position. The controller is configured to receive input from the infra-red sensor that is indicative of a temperature measured by the infra-red sensor. The controller is configured to transmit to an output device an output that is indicative of the temperature measured by the infra-red sensor.

In yet another aspect, there is provided a temperature scanner for a human subject, having a head, and a left eye and a right eye. The temperature scanner includes a housing an infra-red sensor, a left reflector and a right reflector and a controller. The infra-red sensor is mounted to the housing and disposed to cover a field of view. The left and right reflectors are disposed in or on the housing, and provide a reflection of the human subject. The left and right reflectors are spaced apart by a predetermined distance and being sized such that when the head of the human subject moves to a predetermined range of distances, angles and heights relative to the left and right reflectors the subject can see substantially only the subject's left eye in the left reflector and substantially only the subject's right eye in the right reflector, a selected area of the human subject is within the field of view. The controller is configured for sending an output indicative of a temperature reading taken by the infra-red sensor to an output device.

In yet another aspect, there is provided a temperature scanner for measuring a temperature of a selected area on a human subject having a first eye and a second eye. The temperature scanner includes an infra-red sensor that has a selected field of view so as to detect a temperature of the selected area of the human subject when the selected area is positioned to completely fill the field of view, and further includes a first reflective surface region and a second reflective surface region, which resemble a first eyepiece and a second eyepiece from a pair of glasses. The first reflective surface region and the second reflective surface region are positioned relative to the infra-red sensor such that positioning the first eye of the human subject at a selected first eye position and positioning the second eye of the human subject at a selected second eye position positions the selected area of the human subject to completely fill the field of view. A controller is provided and is configured to receive input from the infra-red sensor that is indicative of a temperature measured by the infra-red sensor. The controller is configured to transmit to an output device an output that is indicative of the temperature measured by the infra-red sensor.

### BRIEF DESCRIPTIONS OF THE DRAWINGS

For a better understanding of the embodiment(s) described herein and to show more clearly how the embodiment(s) may be carried into effect, reference will now be made, by way of example only, to the accompanying drawings in which:

FIG. 1 is an elevation view of station with a non-contact temperature scanner in accordance with an embodiment of the present disclosure.

FIG. 2 is a perspective view of a head of a human subject.

FIG. 3 is a perspective view of the temperature scanner shown in FIG. 1.

FIG. 4 is a perspective view of elements inside the temperature scanner shown in FIG. 1.

FIG. 5 is a perspective sectional view a portion of the temperature scanner shown in FIG. 1.

FIG. 6 is an overhead plan view of an arrangement of first and second reflective surface regions for the temperature scanner shown in FIG. 1, wherein the first and second reflective surface regions are both concave.

FIG. 7 is an overhead plan view of an alternative arrangement of first and second reflective surface regions for the temperature scanner shown in FIG. 1, wherein the first and second reflective surface regions are both concave.

FIG. 8 is an overhead plan view of another alternative arrangement of first and second reflective surface regions for the temperature scanner shown in FIG. 1, wherein the first reflective surface region is concave, and the second reflective surface region is convex.

FIG. 9 is an overhead plan view of yet another alternative arrangement of first and second reflective surface regions for the temperature scanner shown in FIG. 1, wherein the first reflective surface region is concave, and the second reflective surface region is planar.

FIG. 10 is an overhead plan view of yet another alternative arrangement of first and second reflective surface regions for the temperature scanner shown in FIG. 1, wherein the first reflective surface region is concave, and the second reflective surface region is planar.

FIG. 11 is a perspective view of an embodiment of the temperature scanner shown in FIG. 1, in which the first and second reflective surface regions are part of a single reflector.

FIG. 12 is a sectional plan view of the temperature scanner shown in FIG. 11.

FIG. 13 is a perspective view of the temperature scanner shown in FIG. 1, with another set of first and second reflective surface regions which are both planar.

FIG. 14 is an overhead plan view of the first and second reflective surface regions from the temperature scanner shown in FIG. 13.

Unless otherwise specifically noted, articles depicted in the drawings are not necessarily drawn to scale.

### DETAILED DESCRIPTION

For simplicity and clarity of illustration, where considered appropriate, reference numerals may be repeated among the Figures to indicate corresponding or analogous elements. In addition, numerous specific details are set forth in order to provide a thorough understanding of the embodiment or embodiments described herein. However, it will be understood by those of ordinary skill in the art that the embodiments described herein may be practiced without these specific details. In other instances, well-known methods, procedures and components have not been described in detail so as not to obscure the embodiments described herein. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description. It will also be noted that the use of the term “a” will be understood to denote “at least one” in all instances unless explicitly stated otherwise or unless it would be understood to be obvious that it must mean “one”.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

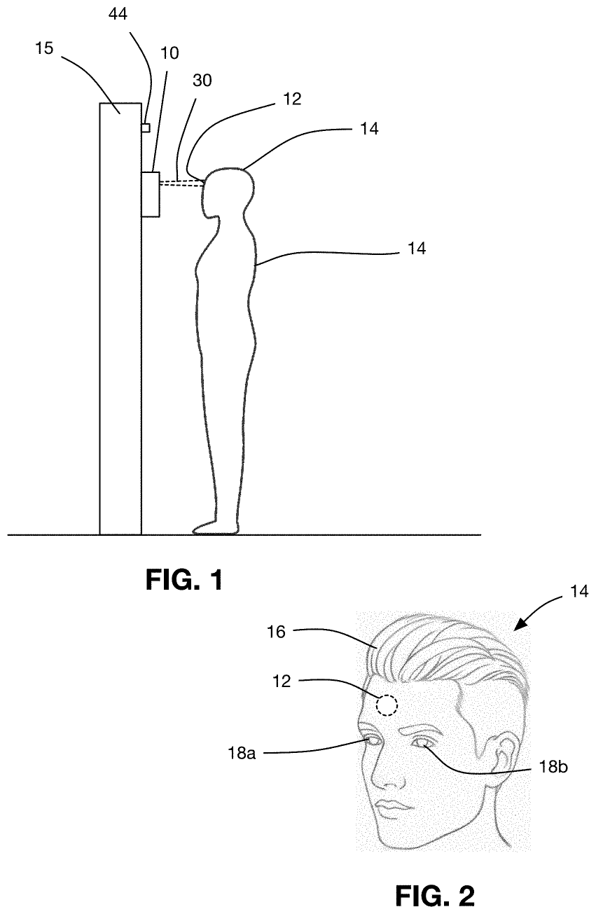

Reference is made to FIG. 1, which shows a temperature scanner 10 for measuring a temperature of a selected area 12 on a human subject 14. The temperature scanner 10 may be set up at a station at an entrance to a business, such as a factory, an office suite, a store, or any other suitable business, in order to determine if the human subject 14 has an elevated temperature and is not permitted to enter the business, or has a non-elevated temperature and is permitted to enter the business. Alternatively, the station with the temperature scanner 10 may be set up simply so that people (e.g. company employees) may voluntarily take their own temperature using a non-contact process so that they can take appropriate action if they determine their temperature is elevated. The station is shown at 15, and is represented in FIG. 1 as a simple wall, or stand upon which the temperature scanner 10 is mounted.

The aforementioned selected area 12 on the human subject 14 may be any suitable selected area, such as, for example, a region in the middle of the forehead of the human subject 14, as shown more clearly by way of a dashed outline in FIG. 2. As can be seen in FIG. 2, the human subject has a head 16, and has a first eye 18a and a second eye 18b, on the head 16.

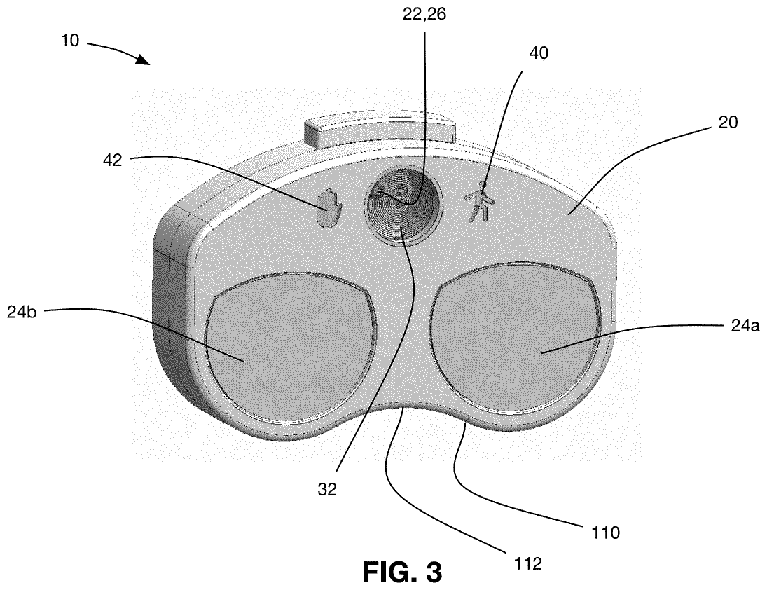

The temperature scanner 10 is configured to take a measurement of the human subject 14 without direct contact. Referring to FIG. 3, the temperature scanner 10 includes a housing 20, an infra-red sensor 22, a controller (FIG. 3) 23, a first reflective surface region 24a and a second reflective surface region 24b. The infra-red sensor 22 may be any suitable type of infra-red sensor, such as a thermopile 26. The thermopile 26 has a field of view 30 (represented by dashed lines) which may increase in size with distance away from the thermopile 26. The thermopile 26 will measure the temperature of whatever is in the field of view 30. In order to limit the size of the field of view 30, the temperature scanner 10 may include a lens 32 in front of the thermopile 26 that focuses incoming infra-red waves. The lens 32 may be any suitable type of lens, such as a Fresnel lens, as shown in FIG. 3.

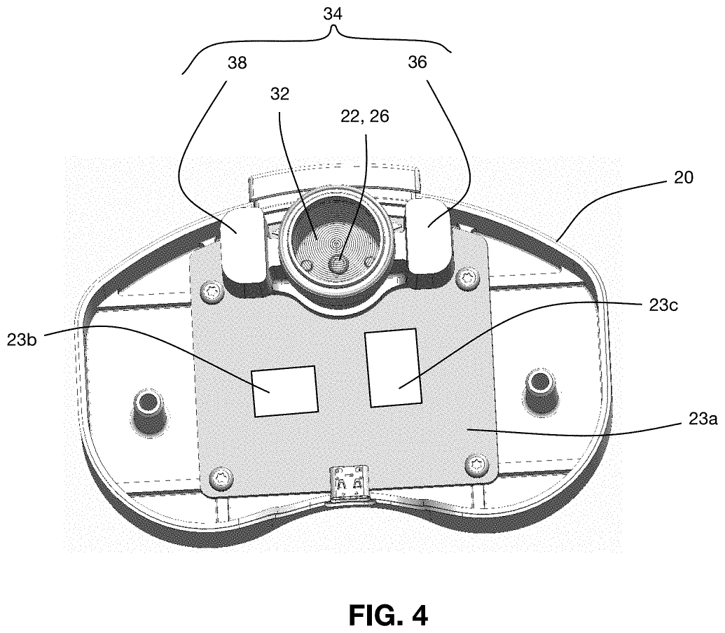

The controller 23 is configured to receive input from the infra-red sensor 22 that is indicative of a temperature measured by the infra-red sensor 22. The controller 22 is configured to transmit to an output device 34 an output that is indicative of the temperature measured by the infra-red sensor 22. The controller 23 includes a PCB 23a, with a processor 23b and a memory 23c thereon. The memory 23c holds code that is executed by the processor 23b and data that is used during the execution of the code. The positions of the processor 23b and the memory 23c shown in FIG. 4 are examples only—the processor 23b and the memory 23c may be positioned in any other suitable positions.

The output device 34 may include a first light-emitting element 36 and a second light emitting element 38. The first light emitting element 36 is positioned behind a first output indicium 40 (FIG. 3), which may be used to indicate that the temperature of the human subject 14 is not elevated. In certain embodiments, the first output indicium 40 is in the form of a person walking, and may be coloured green, so as to indicate that the human subject 14 may therefore proceed past the station 15. The second light-emitting element 38 is positioned behind a second output indicium 42 (FIG. 3), which may be used to indicate that the temperature of the human subject 14 is elevated. In certain embodiments, the output indicium 42 is in the form of a hand, and may be coloured red, so as to indicate that the human subject 14 may not proceed past the station 15.

Different regions on the exterior of the human body have different temperatures. Accordingly, it is helpful, when measuring the temperature of many people, to measure a generally consistent region of their bodies, to avoid having to account for the natural temperature differences that are present on different regions of the exterior of the human body. In order to do this, the temperature scanner 10 is configured to help each human subject 14 to position themselves such that the selected area 12 of each one is positioned to completely fill the field of view 30. By completely filling the field of view 30 of the thermopile 26, the thermopile 26 does not inadvertently receive infra-red waves from both the human subject 14 and from whatever else is in the field of view 30. This helps to ensure that the temperature measured by the thermopile 26 accurately reflects the temperature of the human subject.

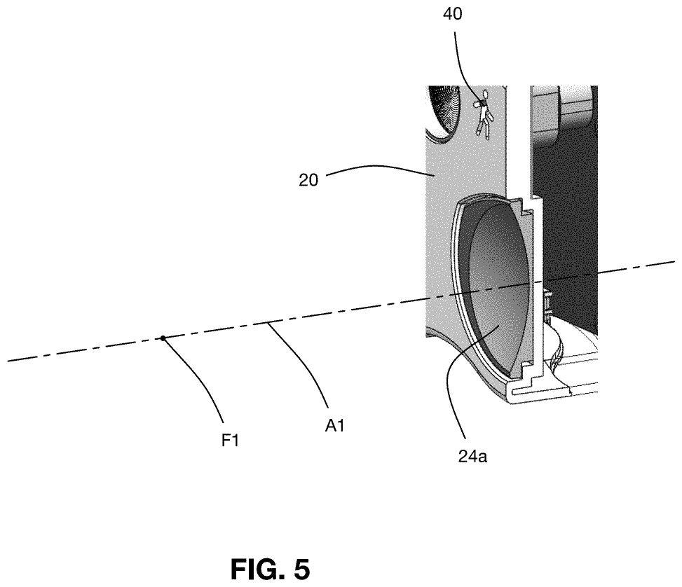

The first reflective surface region 24a is shown more clearly in FIG. 5. In the embodiment shown in FIG. 5, the first reflective surface region 24a is concave, has a first principal axis A1, and has a first focal point F1, along the first principal axis A1 and a first focal length FL1. In the embodiment shown in FIG. 5, the second reflective surface region 24b is concave, has a second principal axis A2, and has a second focal point F2, along the second principal axis A2, and a second focal length FL2. It will be noted that in other embodiments, one or both of the first and second reflective surface regions 24a and 24b are not concave.

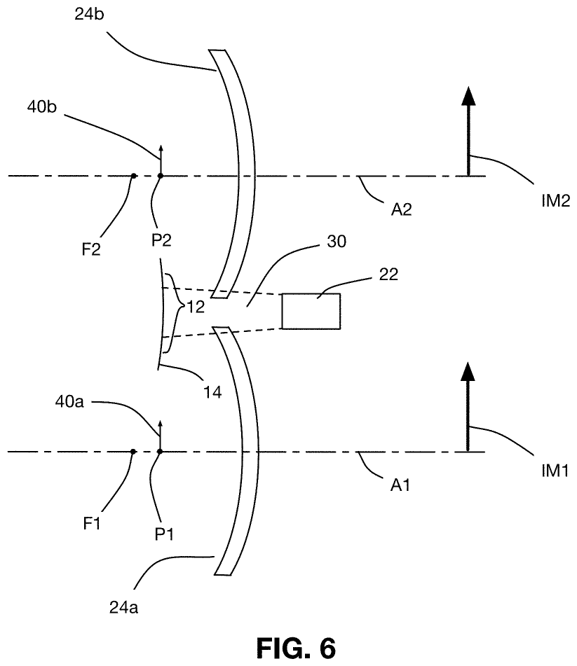

FIG. 6 shows an overhead view of the first and second reflective surface regions 24a and 24b from the embodiment shown in FIG. 5. In FIG. 6, the eyes 18a and 18b of the human subject 14 are represented by a first arrow 40a and a second arrow 40b, respectively. The first and second reflective surface regions 24a and 24b may be positioned relative to the infra-red sensor 22, such that positioning the first eye (first arrow 40a) of the human subject 14 at a selected first eye position P1, and positioning the second eye (second arrow 40b) of the human subject 14 at a selected second eye position P2 positions the selected area 12 of the human subject 14 to completely fill the field of view 30. In the example shown in FIG. 6, the selected first eye position F1 is axially between the first focal point F1 and the first reflective surface region 24a. In the example shown in FIG. 6, where the second reflective surface region 24b is concave, the selected second eye position P2 may be selected to be axially between the first focal point F1 and the first reflective surface region 24a.

It will be noted that FIG. 6 is a schematic representation and is therefore not to scale. In practice, the first focal length FL1 may be about 8 inches (about 20 cm). The second focal length FL2 in FIG. 6 may be the same as the first focal length FL1.

During positioning of the head 15 it will be noted that, as the human subject 14 approaches the position in which their first and second eyes 18a and 18b are in the selected first and second eye positions P1 and P2, the first eye 18a of the human subject 14 can see a reflected image IM1 of the first eye 18a in the first reflective surface region 24a and the second eye 18b of the human subject 14 can see a reflected image IM2 of the second eye 18b in the second reflective surface region 24b. Furthermore, in the selected first eye position P1, the first eye 18a may optionally be able to see a reflected image IM1 of itself, and in the selected second eye position P2, the second eye 18b may optionally be able to see a reflected image IM2 of itself.

In the example shown in FIG. 6, in order to position the first and second eyes 18a and 18b in the selected first and second eye positions P1 and P2, the following method may be used.

The human subject 14 may move their head 15 until they can see reflected images IM1 and IM2 of their first and second eyes 18a and 18b. If the reflected images IM1 and IM2 are inverted, it is because their first and second eyes 18a and 18b are farther from the first and second reflective surface regions 24a and 24b than their respective first and second points F1 and F2. The human subject 14 may bring their first and second eyes 18a and 18b progressively closer to the first and second reflective surface regions 24a and 24b, until they reach a point at which they no longer see reflected images IM1 and IM2. This means that their eyes 18a and 18b are approximately at the focal points F1 and F2. Continuing to approach the first and second reflective surface regions 24a and 24b, will eventually result in reflected images IM1 and IM2 that are upright (and virtual, as opposed to the reflected images IM1 and IM2 when the first and second eyes 18a and 18b are out beyond the focal points F1 and F2, which are inverted and real). The points at which the first and second reflected images IM1 and IM2 appear clear (i.e. not blurry) while the human subject's first and second eyes 18a and 18b approach the first and second reflective surface regions 24a and 24b, may be the selected first and second eye positions P1 and P2. When the first and second eyes 18a and 18b are in the positions P1 and P2 the selected area 12 of the human subject 14 fills the field of view 30 of the infra-red sensor 22.

It will be noted that this is but an example of where the selected first and second eye positions P1 and P2 can be. In an alternative embodiment, the selected first and second eye positions P1 and P2 may be at the focal points F1 and F2, respectively. Accordingly, the human subject 14 may bring their first and second eyes 18a and 18b progressively closer to the first and second reflective surface regions 24a and 24b until there is no longer a reflected image IM1 and IM2 of their first and second eyes 18a and 18b, at which point the first and second eyes 18a and 18b are approximately at the first and second focal points F1 and F2, respectively and therefore are at the selected first and second eye positions P1 and P2.

Once the human subject 14 has their eyes in the selected positions P1 and P2, the human subject 14 may trigger the controller 23 to activate the infra-red sensor 22 by any suitable means. For example, the human subject 14 may wave their arms so as to trip a proximity sensor that is at a suitable height about the housing 20, wherein the proximity sensor (shown at 44 in FIG. 1) sends a signal to the controller 23 in order to initiate actuation of the infra-red sensor 22. In another embodiment, the controller 23 may be programmed (or otherwise configured) to take regular temperature readings from the infra-red sensor 22 and to simply ignore those readings that are too low. Once the human subject 14 has positioned themselves in the field of view 30 of the infra-red sensor 22 the regular temperature readings taken by the infra-red sensor 22 will change as at least some part of its field of view is taken up by the human subject 14. Once the controller 23 receives temperature readings that indicate a change beyond some threshold amount (e.g. 4 degrees Celsius), the controller 23 begins to send output signals to the output device 34. As the human subject 14 reaches the position at which the selected area 12 fills the field of view 30 of the infra-red sensor 22, the controller 23 will be receiving temperature readings that accurately indicate the temperature of the human subject 14. The human subject 14 can then rely on the output (e.g. the illuminated ‘person walking’ icon 40 or the illuminated hand icon 42, as the case may be) as being representative of the temperature of the human subject 14.

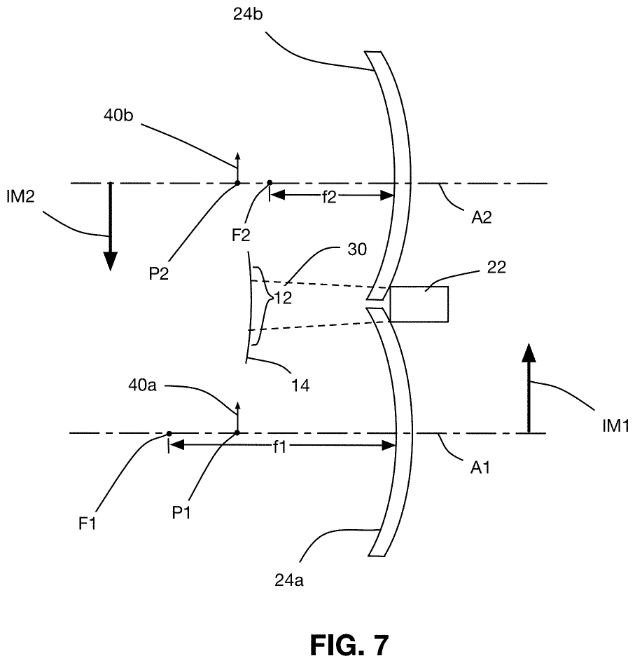

FIG. 7 shows an alternative embodiment of the temperature scanner 10. In the embodiment shown in FIG. 7, the human subject 14 uses a different way than in the embodiment shown in FIG. 6, of determining when they are in the correct position such that their first and second eyes 18a and 18b are in the selected first and second eye positions P1 and P2 such that the selected area 12 fills the field of view 30 of the infra-red sensor 22. In the embodiment shown in FIG. 7, the first reflective surface region 24a has a first focal length f1, and the second reflective surface region 24b has a second focal length f2 that is different than the first focal length f1. The selected first and second eye positions P1 and P2 are selected such that P1 is positioned axially between the first focal point F1 and the first reflective surface region 24a, and P2 is positioned axially farther out from the second reflective surface region 24b than is the second focal point F2. As a result, the first reflected image IM1 is real and inverted, and the second reflected image IM2 is virtual and upright. When the human subject reaches the selected first and second eye positions, the first reflected image IM1 and the second reflected image IM2 are the same size as one another, though they are inverted relative to one another. Therefore, the human subject 14 can determine that they have positioned their first and second eyes 18a and 18b in the selected first and second eye positions P1 and P2 when they see images that are the same size, albeit inverted from one another.

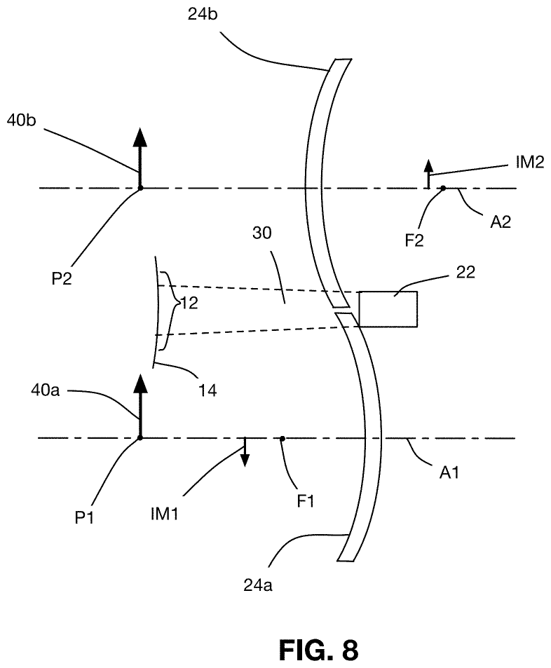

FIG. 8 shows an alternative embodiment of the temperature scanner 10. In the embodiment shown in FIG. 8, the human subject 14 uses another different way than in the embodiments shown in FIGS. 6 and 7, of determining when they are in the correct position such that their first and second eyes 18a and 18b are in the selected first and second eye positions P1 and P2 such that the selected area 12 fills the field of view 30 of the infra-red sensor 22. In the embodiment shown in FIG. 8, the first reflective surface region 24a is concave, as before, but the second reflective surface region 24b is convex. As a result, the reflected image IM2 is smaller than the original object (the second eye 18b of the human subject 14, represented as the second arrow 40b), and is upright (and virtual). When the first eye as represented by the arrow 40a is positioned more than two times the focal length away from the first reflective surface region 24a, the reflected image IM1 is also smaller than the original object (i.e. the first eye 18a as represented by the first arrow 40a), though the reflected image IM1 is inverted relative to the original object. Thus, by selecting the first and second eye positions P1 and P2 appropriately, when the first and second eyes 18a and 18b are in the selected first and second eye positions P1 and P2, the first and second reflected images IM1 and IM2 will be the same size as one another, though the first reflected image IM1 will be inverted. This makes it relatively easy for the human subject 14 to determine when they are in the correct position in order for the selected area 12 to file the field of view 30 of the infra-red sensor 22.

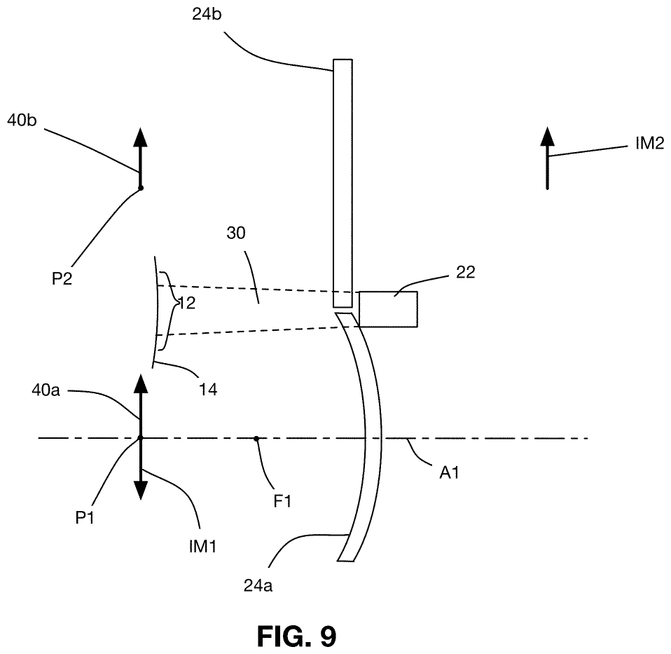

FIG. 9 shows an alternative embodiment of the temperature scanner 10. In the embodiment shown in FIG. 9, the human subject 14 uses another different way than in the embodiments shown in FIGS. 6-8, of determining when they are in the correct position such that their first and second eyes 18a and 18b are in the selected first and second eye positions P1 and P2 such that the selected area 12 fills the field of view 30 of the infra-red sensor 22. In the embodiment shown in FIG. 9, the first reflective surface region 24a is concave, as before, but the second reflective surface region 24b is planar. As a result, the reflected image IM2 is the same size as the original object (the second eye 18b of the human subject 14, represented as the second arrow 40b), and is upright (and virtual). By selecting the focal point F1 of the first reflective surface region 24a such that the selected first eye position is at two times the focal length, the reflected image IM1 is the same size as the original object (the first eye 18a as represented by the first arrow 40a). Thus, when the first and second eyes 18a and 18b are in the selected first and second eye positions P1 and P2, the first and second reflected images IM1 and IM2 will be the same size as one another, though the first reflected image IM1 will be inverted. This again makes it relatively easy for the human subject 14 to determine when they are in the correct position in order for the selected area 12 to file the field of view 30 of the infra-red sensor 22.

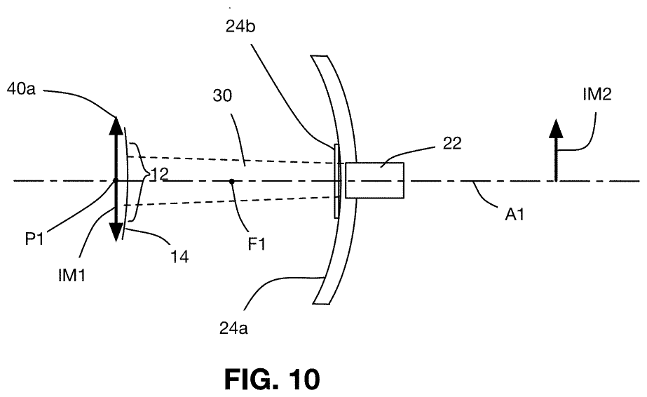

FIG. 10 shows a variant of the temperature scanner 10 shown in FIG. 9. In the embodiment shown in FIG. 9, in which the second reflective surface region 24b is planar and is surrounded by the first reflective surface region 24a.

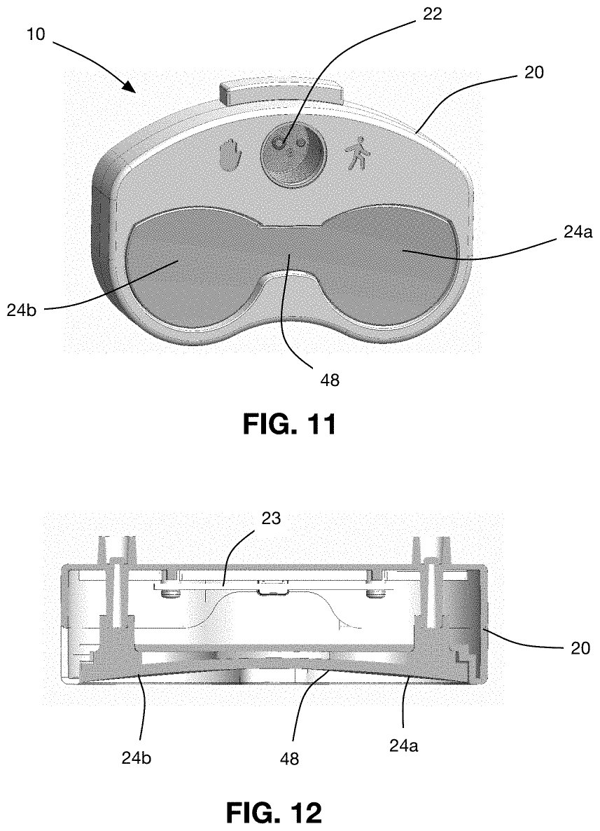

In the embodiments shown in FIGS. 5-10, the first and second reflective surface regions 24a and 24b have been provided on separate first and second reflectors shown at 46a and 46b. By contrast, FIG. 11 shows an example of the temperature scanner 10 in which the first and second surface regions 24a and 24b are both part of a single reflector 48.

FIG. 12 is a sectional view of the temperature scanner 10 shown in FIG. 11, showing more clearly the concave curvature of the single reflector 48.

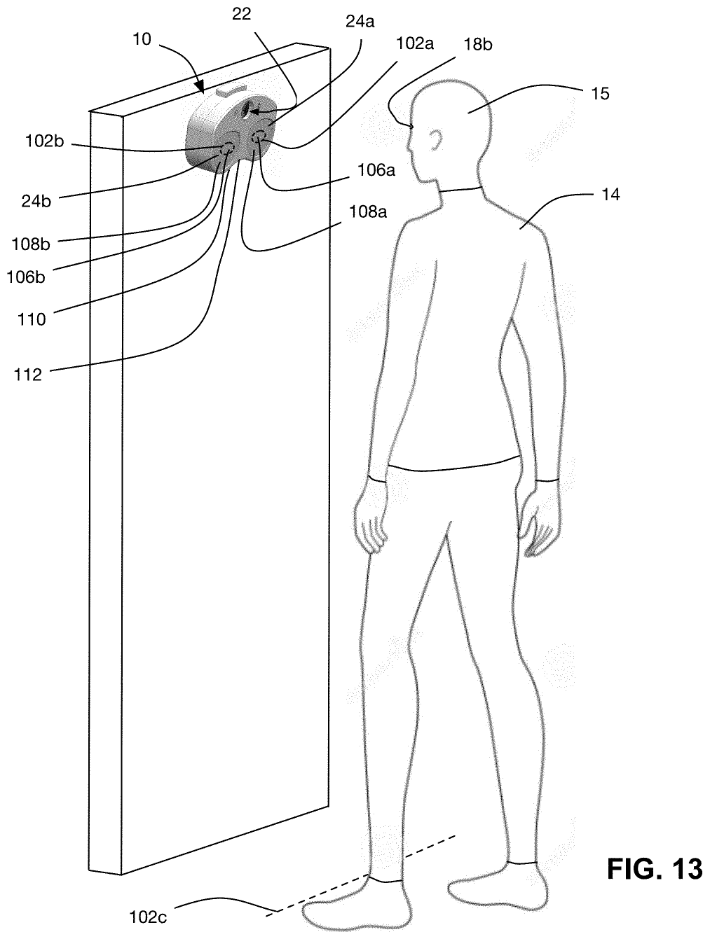

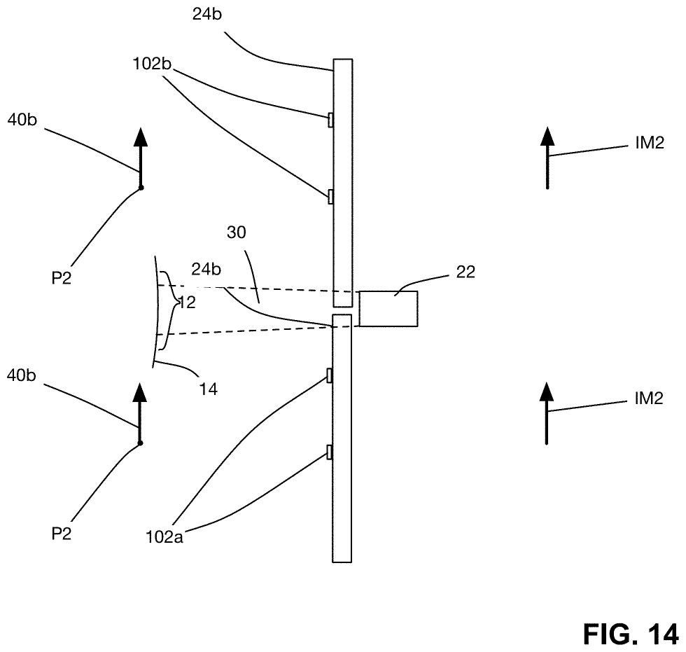

FIG. 13 is a perspective view of the temperature scanner 10 configured such that the first and second reflective surface regions 24a and 24b are both planar. The first and second reflective surface regions 24a and 24b are shown as being provided on separate first and second reflectors, however, they could alternatively be part of a single reflector. As with the embodiments shown in FIGS. 5-10, the first reflective surface region 24a and the second reflective surface region 24b are positioned relative to the infra-red sensor 22, such that positioning the first eye 18a (as represented by arrow 40a) of the human subject 14 at a selected first eye position P1 and positioning the second eye 18b (as represented by arrow 40a) of the human subject 14 at a selected second eye position P2 positions the selected area 12 of the human subject 14 to completely fill the field of view 30. At the selected first eye position P1 the first eye 18a of the human subject 14 can see a reflected image IM1 of the first eye 18a of the human subject 14 in the first reflective surface region 24a, and at the selected second eye position the second eye 18b of the human subject can see a reflected image IM2 of the second eye 18b of the human subject 14 in the second reflective surface region 24b. A set of first positional indicia 102a and a set of second positional indicia 102b are positioned relative to the first and second reflective surface regions, 24a and 24b, such that, positioning of the first and second eyes 18a and 18b of the human subject 14 so as to align the first and second reflected images IM1 and IM2 with the first and second positional indicia 102a and 102b positions the first eye 18a of the human subject 14 in the selected first eye position P1 and positions the second eye 18b of the human subject 14 in the selected second eye position P2, particularly when the human subject is already at the correct distance from the infra-red sensor 22. In order to position the human subject 14 at the correct distance from the infra-red sensor 22, there may also be provided third positional indicia 102c. in the embodiment shown, the third indicia 102c include a line on the floor on which the human subject 14 is standing. The third indicia 102c may also be referred to as distance indicia, since they are used to help position the human subject 14 at the selected distance from the infra-red sensor 22.

The first and second indicia 102a and 102b may be first and second ovals 104a and 104b that are printed on or painted on the first and second reflective surface regions 24a and 24b, and which are shaped and sized similarly to the first and second eyes of an average person.

The first oval 104a makes up a first perimeter marking on the first reflective surface region 24a. The first perimeter marking surrounds a target portion 106a of the first reflective surface region 24a, and is surrounded by a surrounding portion 108a of the first reflective surface region 24a. Similarly, the second oval 104b makes up a second perimeter marking on the second reflective surface region 24a. The second perimeter marking surrounds a target portion 106b of the second reflective surface region 24b, and is surrounded by a surrounding portion 108b of the second reflective surface region 24b.

Thus, the human subject 14 can position themselves such that their first and second eyes 18a and 18b are aligned with (i.e. positioned inside) the first and second positional indicia 102a and 102b. Providing the surrounding portions 108a and 108b permits the human subject 14 to determine where they are relative to the first and second indicia 102a and 102b and to move their first and second eyes 18a and 18b as needed in order to provide the desired alignment therewith.

The first and second indicia 102a and 102b need not be printed on or painted on the first and second reflective surface regions 24a and 24b. The first and second positional indicia 102a and 102b could be in the form of a first surround 106a and a second surround 106b that form part of the housing 20, and which surround at least a portion of the first reflective surface region 24a and at least a portion of the second reflective surface region 24b, respectively. The human subject 14 may position their head 15 such that the first and second reflected images IM1 and IM2 of their eyes 18a and 18b are generally centered in the first and second reflective surface regions 24a and 24b.

In the embodiments shown in the figures, it will be noted that the first and second reflective surface regions 24a and 24b generally resemble in size and shape the eyepieces of a pair of glasses, and indeed, the housing 20, particularly the lower contour thereof (shown at 110), resembles the frame of a pair of glasses. Providing this shape instills in the human subject 14 a natural tendency to position their head such that their first and second eyes 18a and 18b. in other words, the human subject 14 will intuitively know how to orient and position themselves relative to the temperature scanner 10 because of the resemblance of the first and second reflective surface regions 24a and 24b to the eyepieces of a pair of glasses. What provides the first and second reflective surface regions 24a and 24b this feature is that they are generally (in at least some embodiments) not greater than about 4 inches in width each and not greater than about 4 inches in height each, and may not be greater than about 3 inches in width each and not greater than about 3 inches in height. In some embodiments, the width of each of the first and second reflective surface regions 24 and 24b may be greater than the height of each of the first and second reflective surface regions 24 and 24b, thereby further reinforcing the appearance of being eyepieces from a pair of glasses.

The lower contour 110 may be said to resemble the frame of a pair of glasses by virtue of having a notch 112 that is positioned between the first and second reflective surface regions 24a and 24b. The notch 112 is generally shallow in the lower contour 110 but is still suggestive of the portion of the frame of a pair of glasses that fits over the nose of the wearer.

The temperature scanner 10 may be used for a variety of applications. For example, the temperature scanner 10 may be incorporated into a vehicle to permit the user (i.e. the human subject 14) to test themselves before driving or traveling somewhere. The temperature scanner 10 may be incorporated into the B- or C-pillar of a vehicle such as a taxi in order for a potential passenger to test their temperature before being permitted to enter the vehicle. The temperature scanner 10 could be interlocked with a barrier that prevents entry into a business unless the temperature of the human subject is not elevated. Other applications may also exist.

Persons skilled in the art will appreciate that there are yet more alternative implementations and modifications possible, and that the above examples are only illustrations of one or more implementations. The scope, therefore, is only to be limited by the claims appended hereto and any amendments made thereto.

What is claimed is:

1. A temperature scanner for measuring a temperature of a selected area on a human subject having a first eye and a second eye, comprising:
an infra-red sensor that has a selected field of view so as to detect a temperature of the selected area of the human subject when the selected area is positioned to completely fill the field of view;
a first reflective surface region and a second reflective surface region, wherein the first reflective surface region is concave, has a first principal axis, and has a first focal point along the first principal axis,
wherein the first reflective surface region and the second reflective surface region are positioned relative to the infra-red sensor such that positioning the first eye of the human subject at a selected first eye position and positioning the second eye of the human subject at a selected second eye position positions the selected area of the human subject to completely fill the field of view; and
a controller configured to receive input from the infra-red sensor that is indicative of a temperature measured by the infra-red sensor, wherein the controller is configured to transmit to an output device an output that is indicative of the temperature measured by the infra-red sensor.

2. The temperature scanner as claimed in claim 1, wherein the selected first eye position is axially between the first focal point and the first reflective surface region.

3. The temperature scanner as claimed in claim 1, wherein the second reflective surface region is concave, has a second principal axis, and has a second focal point along the second principal axis.

4. The temperature scanner as claimed in claim 3, wherein the first principal axis and the second principal axis are coincident with one another, and the first focal point and the second focal point are in the same position as one another.

5. The temperature scanner as claimed in claim 3, wherein the first principal axis and the second principal axis are offset from and parallel to one another.

6. The temperature scanner as claimed in claim 1, wherein the second reflective surface region is flat, and wherein at the selected second eye position, the second eye of the human subject can see a reflected image of the second eye of the human subject in the second reflective surface region.

7. The temperature scanner as claimed in claim 6, wherein at the selected first eye position the first eye of the human subject can see a reflected image of the first eye of the human subject in the first reflective surface region,
and wherein, at the selected first eye position and at the selected second eye position, the reflected image of the first eye and the reflected image of the second eye are approximately the same size as one another.

8. The temperature scanner as claimed in claim 1, wherein the second reflective surface region is concave,
wherein at the selected first eye position the first eye of the human subject can see a reflected image of the first eye of the human subject in the first reflective surface region, and at the selected second eye position, the second eye of the human subject can see a reflected image of the second eye of the human subject in the second reflective surface region,
wherein, at the selected first eye position and at the selected second eye position, the reflected image of the first eye and the reflected image of the second eye are approximately the same size as one another.

9. The temperature scanner as claimed in claim 8, wherein the first reflective surface region has a first focal length and the second reflective surface region has a second focal length that is different than the first focal length, such that, at the selected first eye position the first reflected image is real and inverted, and at the selected second eye position the second reflected image is virtual and upright.

10. The temperature scanner as claimed in claim 1, wherein the second reflective surface region is convex,
wherein at the selected first eye position the first eye of the human subject can see a reflected image of the first eye of the human subject in the first reflective surface region, and at the selected second eye position, the second eye of the human subject can see a reflected image of the second eye of the human subject in the second reflective surface region,
wherein, at the selected first eye position and at the selected second eye position, the reflected image of the first eye and the reflected image of the second eye are approximately the same size as one another.

11. The temperature scanner as claimed in claim 1, wherein the first reflective surface region and the second reflective surface region are both part of a single reflector.

12. The temperature scanner as claimed in claim 1, wherein the first reflective surface region is part of a first reflector and the second reflective surface region is part of a second reflector that is separate from the first reflector.

13. A temperature scanner for measuring a temperature of a selected area on a human subject having a first eye and a second eye, comprising:
an infra-red sensor that has a selected field of view so as to detect a temperature of the selected area of the human subject when the selected area is positioned to completely fill the field of view;
a first reflective surface region and a second reflective surface region, wherein the first reflective surface region and the second reflective surface region are positioned relative to the infra-red sensor, such that positioning the first eye of the human subject at a selected first eye position and positioning the second eye of the human subject at a selected second eye position positions the selected area of the human subject to completely fill the field of view, wherein, at the selected first eye position the first eye of the human subject can see a reflected image of the first eye of the human subject in the first reflective surface region, and at the selected second eye position the second eye of the human subject can see a reflected image of the second eye of the human subject in the second reflective surface region;
a set of first positional indicia and a set of second positional indicia positioned relative to the first and second reflective surface regions, wherein, positioning of the first and second eyes of the human subject so as to align the first and second reflected images with the first and second positional indicia positions the first eye of the human subject in the selected first eye position and positions the second eye of the human subject in the selected second eye position; and
a controller configured to receive input from the infra-red sensor that is indicative of a temperature measured by the infra-red sensor, wherein the controller is configured to transmit to an output device an output that is indicative of the temperature measured by the infra-red sensor.

14. The temperature scanner as claimed in claim 13, wherein the first positional indicia include a first surround that surrounds at least a portion of the first reflective surface region and the second positional indicia include a second surround that surrounds at least a portion of the second reflective surface region.

15. The temperature scanner as claimed in claim 13, wherein the first positional indicia include a first perimeter marking on the first reflective surface region, wherein the first perimeter marking surrounds a target portion of the first reflective surface region, and is surrounded by a surrounding portion of the first reflective surface region.

16. The temperature scanner as claimed in claim 13, wherein the first reflective surface region and the second reflective surface region are both part of a single reflector.

17. The temperature scanner as claimed in claim 13, wherein the first reflective surface region is part of a first reflector and the second reflective surface region is part of a second reflector that is separate from the first reflector.

18. A temperature scanner for measuring a temperature of a selected area on a human subject having a first eye and a second eye, comprising:
a housing;
an infra-red sensor that has a selected field of view so as to detect a temperature of the selected area of the human subject when the selected area is positioned to completely fill the field of view;
a first reflective surface region and a second reflective surface region, wherein the first reflective surface region and the second reflective surface region resemble a first eyepiece and a second eyepiece from a pair of glasses,
wherein the first reflective surface region and the second reflective surface region are positioned relative to the infra-red sensor such that positioning the first eye of the human subject at a selected first eye position and positioning the second eye of the human subject at a selected second eye position positions the selected area of the human subject to completely fill the field of view; and
a controller configured to receive input from the infra-red sensor that is indicative of a temperature measured by the infra-red sensor, wherein the controller is configured to transmit to an output device an output that is indicative of the temperature measured by the infra-red sensor.

19. The temperature scanner as claimed in claim 18, wherein the housing has a lower contour that includes a notch positioned between the first and second reflective surface regions.

20. The temperature scanner as claimed in claim 18, wherein the first and second reflective surface regions are each less than about 4 inches in width and less then about 4 inches in height.
